(19) United States
(12) Patent Application Publication
(10) Pub. No.: US 2004/0116963 A1
(43) Pub. Date: Jun. 17, 2004

(54) Needle-suture combinations and methods of use

(76) Inventor: Omar M Lattouf, Atlanta, GA (US)

Correspondence Address: Christopher B Linder, Thomas Kayden Horstemeyer & Risley, Suite 1750, 100 Galleria Parkway NW, Atlanta, GA, 30339-5948, US

(21) Appl. No.: 10/472,972

(22) PCT Filed: Jan. 24, 2003
(86) PCT No.: PCT/US03/02367

(30) Foreign Application Priority Data
Jan. 26, 2002 (US) 60352184

(51) Int. Cl. A61B 17/06

(52) U.S. Cl. 606/224

(57) ABSTRACT

Needle-suture combinations are disclosed that include two surgical needles, each including an end having a suture-receiving aperture, and two sutures attached to the ends of each of the two needles. Also disclosed are methods for using the needle-suture combination, the method including passing through a first tissue and a second tissue two needle-suture combinations each having first and second needles connected to first and second sutures; pulling the sutures therethrough; disconnecting the needles from the sutures, thereby providing free ends on the sutures; tying the free ends of the first sutures of the first needle-suture combination together; and tying the free end of the second suture of the first needle-suture combination with the second suture of the second needle-suture combination. With the apparatus and/or method, elements joined together may form a more hemostatic seal.

### CLAIM OF PRIORITY

This application claims priority to copending U.S. provisional application entitled, “Double stitch: A new suture-needle combination with two surgical needles attached together by two strings of sutures instead of one. Prepared with or without pledget,” having Ser. No. 60/352,184 filed Jan. 26, 2002, which is entirely incorporated herein by reference.

### BACKGROUND

1. Field of the Invention

The present invention is generally related to surgical needles and sutures and, more particularly, is related to needle-suture combinations and methods for using needle-suture combinations.

2. Description of Related Art

For many years, surgeons have employed needle-suture combinations in which a suture or ligature is attached to the shank end of a needle. An exemplary prior art needle-suture combination is depicted in FIGS. 1A and 1B. The needle-suture combination 10 illustrated in FIG. 1A includes a pair of needles 12 mounted to the ends 13 of the suture 14. A pledget member 16 is mounted to a center portion 17 of the suture 14. The pledget 16 has a top side 18, sides 19 and a bottom side 21. The suture 14 extends or is “woven” through the pledget 16 through suture mounting holes 23, as can be seen more clearly in FIG. 1B. Because the suture 14 attached to each needle 12 is a single suture, one disadvantage is that the suture 14 may not be strong enough in certain applications, or the suture 14 may need to be made of a particularly strong material. Further, patients undergoing anastomosis and heart valve replacement procedures performed using the needle-suture combination 10 may experience leaking of blood and fluid through the stitches formed, particular under high aortic pressures. Problems associated with this complication are detailed below.

An alternative exemplary prior art needle-suture combination is depicted in FIG. 2, and is manufactured by and commercially available from Genzyme Surgical Products Corp., Fall River, Mass., USA, under the name NextStitch™. The needle-suture combination 25 includes ten needles 27 mounted to and connecting the ends 28 of the nine sutures 29, in the configuration shown in FIG. 2. The needles 27 on the two ends of the needle-suture combination 25 are each mounted to one suture 29, while the needles 27 in the middle of the needle-suture combination 25 are each mounted to the two sutures 29. As with the needle-suture combination 10 of FIGS. 1A and 1B, the pledget members 35 are mounted to a center portion of the sutures 29. The pledgets 35 are generally of the configuration of the pledget 16 of FIGS. 1A and 1B. Again, similar to FIGS. 1A and 1B, sutures 29 extend or are “woven” through each respective pledget 35 through suture mounting holes (not shown).

The needle-suture combination 25 is designed for use primarily as a cardiovascular valve suture. While the needle-suture combination 25 was designed in attempt to solve some of the problems and disadvantages presented by the needle-suture combination 10, other problems and disadvantages are associated with its use. The needle-suture combination 25 is conventionally sold in a needle-suture pack in the configuration illustrated in FIG. 2, i.e., ten needles 27 mounted to nine sutures 29 and nine pledgets 35 mounted on the sutures 29. Therefore, if the desired application calls for fewer than nine stitches, or a number of stitches not a multiple of nine, the extra needles and sutures have to be cut off the needle-suture combination 25. Unfortunately, the needle-suture combination 25 can be difficult or confusing to implement in surgery. In using the needle-suture combination 25, a new procedure is involved in the interaction between a surgeon and an assistant or nurse aiding the surgeon. To keep the needles and sutures in order, a large amount of training and practice must be completed before the needle-suture combination 25 can be used. Further, if the needles and sutures get tangled, or out of order, after being threaded through a patient's flesh, they must all be cut out and the surgeon must begin again, wasting operating time and possibly damaging the patient tissue.

Needle-suture combinations, such as those depicted in FIGS. 1A, 1B, and 2 are used in a variety of surgical procedures and, in particular, with vascular surgery. Important types of vascular surgery include replacement or bypassing of a diseased, occluded, or injured artery and heart valve replacement surgery. Arterial replacement or bypass grafting has been performed for many years using open surgical techniques and a variety of prosthetic grafts. These grafts are manufactured as fabrics (often from DACRON® (polyester fibers) or TEFLON® (fluorocarbon fibers)), or are prepared as autografts (from the patient's own tissues) or heterografts (from the tissues of animals) or a combination of tissues, semi-synthetic tissues and/or alloplastic materials. A graft can be joined to the target artery in a number of different positions, including end-to-end, end-to-side, and side-to-side. This attachment between artery and graft, or any two vessels, is known as an “anastomosis.”

The replacement of diseased or damaged heart valves with artificial heart valves is also a relatively common surgical procedure. The replacement of a heart valve is necessary when the native valve becomes sufficiently incompetent such that coronary function is compromised. There are numerous types of conventional artificial heart valves which can be used in such procedures including synthetic mechanical, porcine tissue, cryogenically preserved homografts, and autologous valves from a different position in the patient's own heart.

For a valve replacement surgical procedure to be successful, the junction of the replacement valve and the heart tissue must be hemostatic, i.e., leakage around the sewing ring must not occur. Leakage from an inadequately mounted replacement valve, known as “paravalvular leakage” can result in regurgitation or backflow of blood that could compromise coronary function. Indeed, paravalvular leakage after aortic or mitral valve replacement can be a potentially life-threatening complication. Moderate to severe paravalvular leakage found after weaning from cardiopulmonary bypass is usually a result of technical error (i.e., the stitch pulling through the heart tissue or poor seating or sizing of the heart valve) and necessitates valve removal and repeat replacement. Accordingly, in addition to using a sufficient number of sutures, the sutures must be sufficiently tensioned to prevent leakage. Due to the relatively narrow diameter of the sutures, tensioning can cause a number of complications including suture tearing through tissue, or tissue bunching that inhibits correct seating of the valve on the annulus. In order to distribute the force applied on cardiac tissue by the tensioned suture, it is known to mount various types of buttress materials to suture. One type of conventional buttress is the pledget described above. The pledget is mounted to the suture and assists in preventing tissue tear through.

Continuous, interrupted, or semi-continuous suture techniques have been used to place prosthetic valves for valvular heart disease and are commonly known in the art. The continuous technique has the advantage of tying fewer knots and therefore reduces aortic cross clamp time. The major disadvantage of the continuous technique is the catastrophic complication of prosthetic valve dehiscence in the event of breakage of a single suture. The advantages of the interrupted method include security with multiple sutures anchoring the prosthesis and the everting nature of the suture. The placement of multiple sutures and multiple ties, however, prolongs aortic cross clamp time. The semi-continuous or “hoist” method known in the art combines advantages of both the interrupted and continuous techniques, but has the disadvantage in that a breakage in that a breakage in one suture leads to a leak from the suture line.

The presence of infection, mismatch between the prosthesis and heart tissue, excessive tension on sutures or the heart tissue, and specific technique of suture placement of suture placement or insufficient of sutures may lead to the occurrence of periprosthetic leak. Continuous suture techniques have been found to be associated with a high rate of periprosthetic leak in one study and found to prevent leak in another.

Although the surgical sutures and cardiac surgical procedures of the prior art are adequate for their intended purpose, there is a need in the art for improved needle-suture combinations. In particular, there is a need to provide improved, novel methods of using needle-suture combinations, along with improved uses for the needle-suture combinations that provide consistent and improved fluid-tight interfaces between a replacement heart valve and the tissue in the heart, or between vessels being anastomosized.

### SUMMARY

Embodiments of the present invention provide surgical devices, namely needle-suture combinations, and methods for using the needle-suture combinations.

Briefly described, one embodiment of such a needle-suture combination includes two surgical needles, each including an end having a suture-receiving aperture; and two sutures attached to each of the two needles at the end having a suture-receiving aperture.

One embodiment of a method for using a needle-suture combination includes: passing through a first tissue and a second tissue two needle-suture combinations each having first and second needles connected to first and second sutures; pulling the sutures therethrough; disconnecting the needles from the sutures, thereby providing free ends on the sutures; tying the free ends of the first sutures of the first needle-suture combination together; and tying the free end of the second suture of the first needle-suture combination with the second suture of the second needle-suture combination.

### BRIEF DESCRIPTION OF THE DRAWINGS

Many aspects of the invention can be better understood with reference to the following drawings. The components in the drawings are not necessarily to scale. Moreover like reference numerals designate corresponding parts throughout the several views.

FIG. 1A is a perspective view of a first prior art needle-suture combination.

FIG. 1B is a perspective view of the bottom side of a pledget shown in FIG. 1 with a suture mounted or woven therethrough.

FIG. 2 is a perspective view of a second prior art needle-suture combination.

FIG. 3 is a perspective view of an embodiment of the disclosed needle-suture combination.

FIG. 4A is a top perspective view illustrating a first threading arrangement for the pledget shown in FIG. 3.

FIG. 4B is a bottom perspective view of the threading arrangement of FIG. 4A.

FIG. 5A is a top perspective view illustrating a second threading arrangement of the pledget shown in FIG. 3.

FIG. 5B is a bottom perspective view of the threading arrangement of FIG. 5A.

FIG. 6A is a perspective view of a first knotting arrangement for a suture woven through a pledget.

FIG. 6B is a perspective view of a second knotting arrangement for a suture woven through a pledget.

FIG. 6C is a perspective view of a third knotting arrangement for a suture woven through a pledget.

FIG. 7A is a side view of a first attachment arrangement for a pair of sutures to a needle.

FIG. 7B is a side view of a second attachment arrangement for a pair of sutures to a needle.

FIG. 8 is a top view illustrating a first application of the disclosed needle-suture combination.

FIG. 9 is a side view of FIG. 8 illustrating a first application of the disclosed needle-suture combination.

FIG. 10 is a top view illustrating a second application of the disclosed needle-suture combination.

FIG. 11 is a line graph comparing a leakage rate of a sample aortic anastomosis using the disclosed needle-suture combination with the leakage rate of a sample aortic anastomosis sutured using a common prior art needle-suture combination.

FIG. 12 is a bar graph comparing a leakage rate of a sample aortic anastomosis using the disclosed needle-suture combination with the leakage rate of a sample aortic anastomosis sutured using a common prior art needle-suture combination, at a typical intra-aortic pressure of 10 mmHg.

### DETAILED DESCRIPTION

As identified in the foregoing, needle-suture combinations and methods for using them are not always sufficient to prevent leakage between tissues or vessels being stitched together. In attempting to solve this problem, other needle-suture combinations may be confusing or difficult to use. Therefore, needed are needle-suture combinations that provide a more hemostatic seal between tissues or vessels and that are easy to use.

Disclosed herein are needle-suture combinations and methods of using the needle suture combinations. The disclosed needle-suture combinations provide sutures that allow twice the number of stitches to be formed using the same number of needle pulls through the tissues or vessels, forming a more hemostatic seal between tissues or vessels.

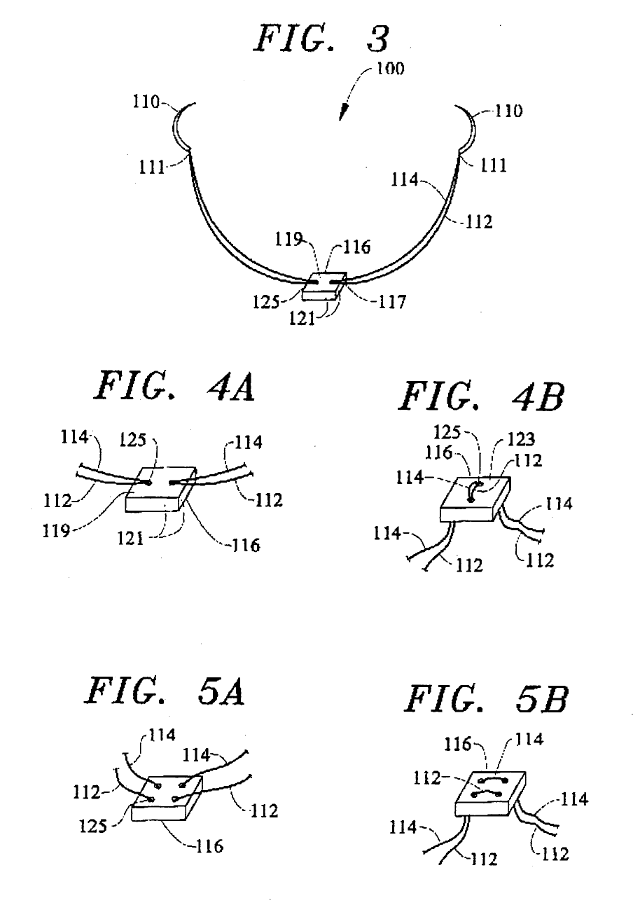

Referring now to the figures, FIG. 3 illustrates an exemplary embodiment of a needle-suture combination 100. The needle-suture combination 100 includes a pair of needles 110 mounted to ends 111 of two sutures 112, 114.

Needles 110 may be fabricated from metals such as stainless steel alloys that have desired characteristics with respect to biocompatibility, strength, and the ability to take a sharp end and/or point when ground and polished. Generally, the needle can be fabricated from a material such as series 300 stainless steel alloy, series 400 stainless steel alloy, or nonferrous alloy, e.g., MP35N alloy. Sutures may be of a wide variety of monofilament and braided suture materials, both absorbable and non-absorbable, e.g., catgut, silk, nylon, polyester, polypropylene, linen, cotton, as well as absorbable synthetic materials such as polymers and copolymers of glycolic and lactic acids. In most anastomosis and cardiovascular procedures it is preferable to use a non-absorbable suture material. In some embodiments the first suture 112 has a different color than suture 114, so that the two sutures 112, 114 are visually distinct from each other in terms of color or some other identifying mode. Advantages of having the two sutures being readily separately identifiable will be described in more detail below.

A pledget member 116 may be positioned on a center portion 117 of the sutures 112, 114, with the sutures 112, 114 being threaded through the pledget 116. The pledget 116 has a top side 119, sides 121, and a bottom side 123 (shown in FIG. 4B). While the pledget 116 shown throughout the figures is substantially square or rectangular in shape, other pledget shapes may be used, for example, circular, elliptical, tubular, and combinations thereof. The pledget 116 is made from soft, pliable biocompatible materials. For example, pledget material may include polytetrafluoroethylene (PTFE), expanded PTFE, polyester, cotton, and combinations thereof.

As can be seen more clearly in FIGS. 4A and 4B the sutures 112, 114 may extend, or be “threaded”, through the pledget 116 through openings 125 formed by the threading process. In this configuration, the pledget 116 is slidably mounted to the sutures 112, 114. Alternatively and/or additionally, the pledget 116 may be fixed in place along the sutures 112, 114 such that it is stationary.

While the pledget 116 of FIGS. 3, 4A, and 4B is shown to have two openings 125, an alternative number of openings 125 may also be formed. FIGS. 5A and 5B depict an alternative threading embodiment for the pledget 116. In this embodiment, more than two openings 125 are formed. In particular, the sutures 112, 114 are threaded through the pledget 116 so as to form two pairs of openings 125. With this manner of threading, the first suture 112 may extend through a first pair of openings 125, and the second suture 114 may extend through a second pair of openings 125.

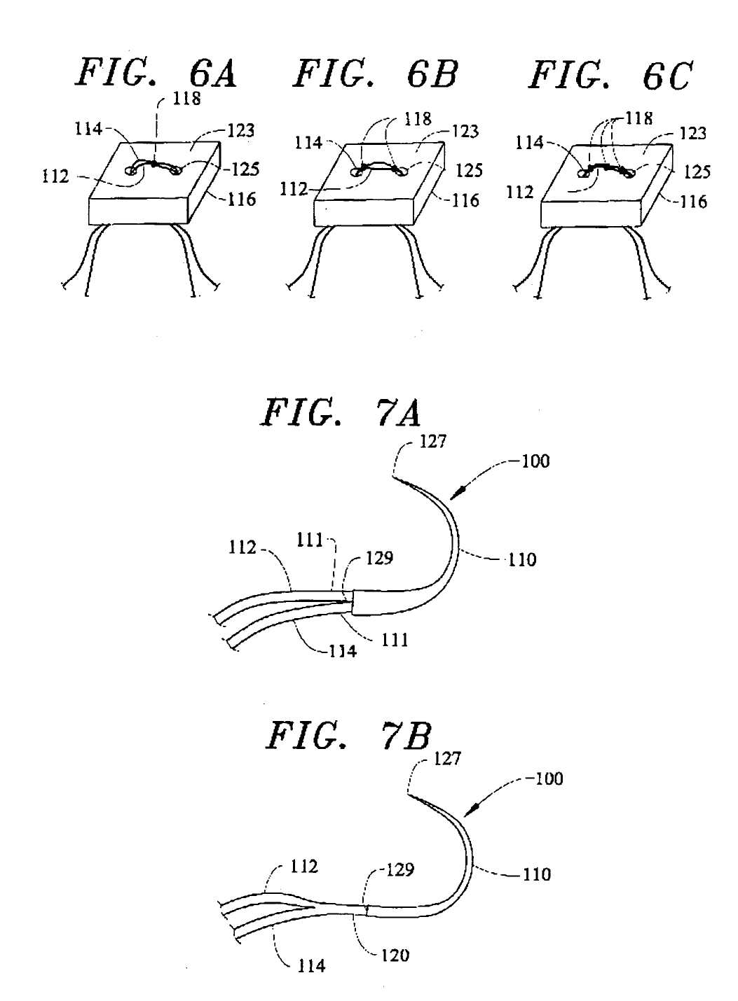

In some cases, it may be preferable to have the sutures 112, 114 include a knot 118 on the bottom side 123 of the pledget 116 when they are threaded through the pledget 116, as shown in FIGS. 6A-6C. FIGS. 6A-6C depict alternative configurations for the placement and number of knots 118. FIG. 6A shows the knot 118 substantially centered between the two openings 125, while FIGS. 6B and 6C show multiple knots 118. In FIG. 6B there are two knots 118, with one knot 118 located adjacent each opening 125. FIG. 6C combines the knot configurations of FIGS. 6A and 6B. Alternative placement of the knots 118 may be used.

While FIGS. 6A-C depict knotted sutures 112, 114 threaded through a single pair of openings 125, the sutures 112, 114 may also be knotted when mounted to the pledget 116 through multiple pairs of openings 125. For example, the configurations of knots 118 in FIGS. 6A-6C can also be accomplished when the sutures 112, 114 are threaded through two pairs of openings 125 (as in the embodiment of FIGS. 5A and 5B). Knot(s) 118 may be advantageous in that they may prevent the sutures 112, 114 from slipping through the openings 125 when the sutures 112, 114 are being tied during surgery. Therefore, knot(s) 118 provide counter-tension on the sutures 112, 114 when a surgeon is pulling on the sutures 112, 114 in order to tie them tightly, and to press the pledget 116 against tissue being sutured (as will be described below in more detail).

Turning now to FIGS. 7A and 7B, exemplary embodiments are shown of the attachment of the needle 110 and the sutures 112, 114 of needle-suture combination 100. The sutures 112, 114 may be attached to the needle 110 in many different manners, including through use of adhesive such as glue or cement, or by ultrasonic welding, shrink-wrap tubing, swaging needle 110 over the ends 111 of the sutures 112, 114, and combinations and equivalents thereof. The needle 110 has two ends, including a pointed tip 127 at one end and a suture-receiving aperture 129 at the other end. In the embodiment of FIG. 7A, both suture ends 111 are inserted into the suture-receiving aperture 129 of the needle 110 and attached by any of the methods described above. To this end, the suture-receiving end of needle 110 may be slightly larger than conventional needles, so as to receive both sutures 112, 114 in the suture-receiving aperture 129. FIG. 7B illustrates an alternative method of attachment of the sutures 112, 114 to the needle 110. In FIG. 7B, the ends 111 of the sutures 112, 114 have been intertwined, or commingled, forming a single end portion 120. The end portion 120 is then inserted into the suture-receiving aperture 129 and attached by any method described above to attach a suture to a needle.

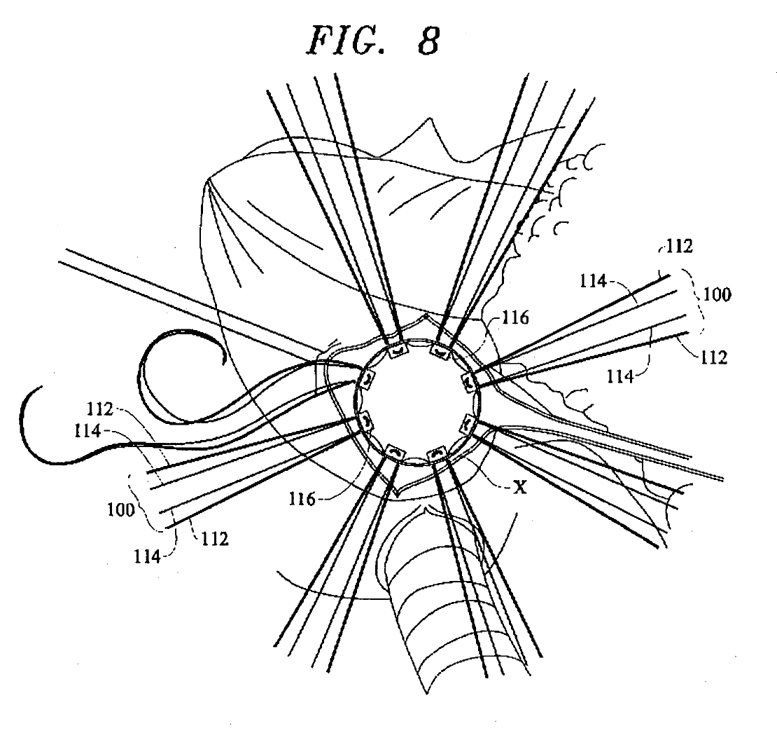

The needle-suture combination 100 described above is particularly useful in procedures involving the anastomosization of vessels and in joining two tissues that are substantially circular or elliptical in shape. For example, the needle-suture combination 100 may be used in heart valve replacement procedures. FIG. 8 illustrates of a heart tissue X with a replacement valve sutured in place using an embodiment of the needle-suture combinations 100. In order to attach a replacement valve in this manner, the surgeon grasps the needle 110 with a needle grasper and inserts the needle 110 through the heart tissue X and then through a portion of the replacement valve, such as a sewing ring (not shown). Next, the surgeon pulls the sutures 112, 114 through the replacement valve such that the top side 119 of the pledget 116 contacts with the heart tissue X. The surgeon then places a number of the needle-suture combinations 100 through the heart tissue X and replacement valve, spacing the needle-suture combinations 100 a sufficient distance apart so as to provide effective hemostatic sealing between the heart tissue X and the replacement valve. This distance will vary with the condition and age of the patient and the individual characteristics of the heart tissue X. Typically, however, this distance is about 1 millimeter (mm) to about 6 mm, more typically about 2 mm to about 5 mm, and preferably about 3 mm to about 4 mm. Although not required, it is preferred that every two adjacent sutures through the heart tissue X and replacement valve be of the same color.

Once a sufficient number of needle-sutures 100 have been threaded through the heart tissue X and the replacement valve, the surgeon slides or “parachutes” the valve onto the heart tissue X to place the valve in the position shown in FIG. 8. At that time, the surgeon cuts the needles 110 from the sutures 112, 114 such that each suture 112, 114 has a free end 111. The needles 110 are usually cut from the sutures 112, 114 prior to sliding the valve into place, but this may also be performed subsequently.

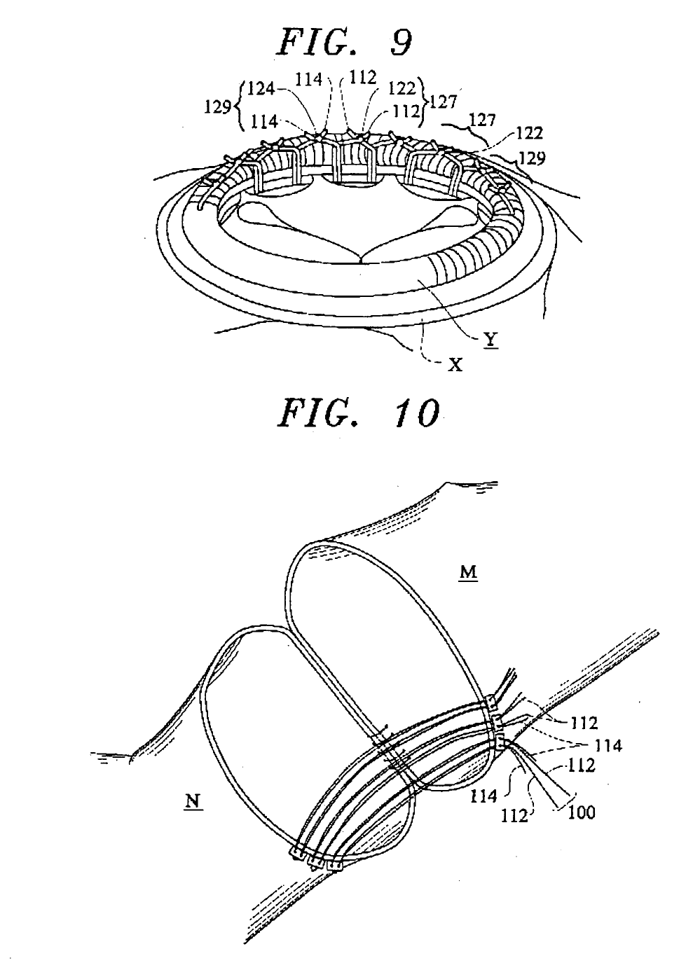

The surgeon then ties or knots the sutures 112, 114 with sufficient tension to mount the valve Y to the heart tissue X in a hemostatic manner, as seen in FIG. 9. Due to the use of the double sutures 112, 114, the first suture 112 is tied to itself (the contiguous suture 112 mounted to the pledget 116) to form a first knot 122. When multiple such knots 122 are tied, a continuous horizontal “mattress” type suture configuration 127 is obtained, as shown in FIG. 9. In addition, the second suture 114 is tied to a different adjacent second suture 114, cut from an adjacent needle-suture combination 100, forming a second knot 124 and creating an additional “running” suture configuration 129 along the ring of valve Y. In situations in which the surgeon uses needle-suture combinations 100 that include a first suture 112 of a first color and a second suture 114 of a second color, tying the knots correctly is simplified. In particular, the surgeon ties sutures of like colors together. Simplification in tying the knots helps avoid the situation where the surgeon may have to cut out the sutures and replace them.

FIG. 10 illustrates a use of the needle-suture combination 100 in an exemplary anastomosis of two vessels M and N. The ends 111 of adjacent first sutures 112 are tied together, and the ends 111 of the second sutures 114, cut from adjacent needle-suture combination 100, are tied together, thereby forming a hemostatic seal around the anastomosized vessels M and N.

Further, while specific examples of methods of using the needle-suture combination 100 given above refer to vessel anastomosis and heart valve replacement procedures, the needle-suture combination 100 can also be used in many other procedures, including vascular grafting, ventricular wall aneurysectomy, coronary revascularization, bowel anastomosis, and suturing of other circular incisions.

Testing the Efficacy of Needle-Suture Combination 100

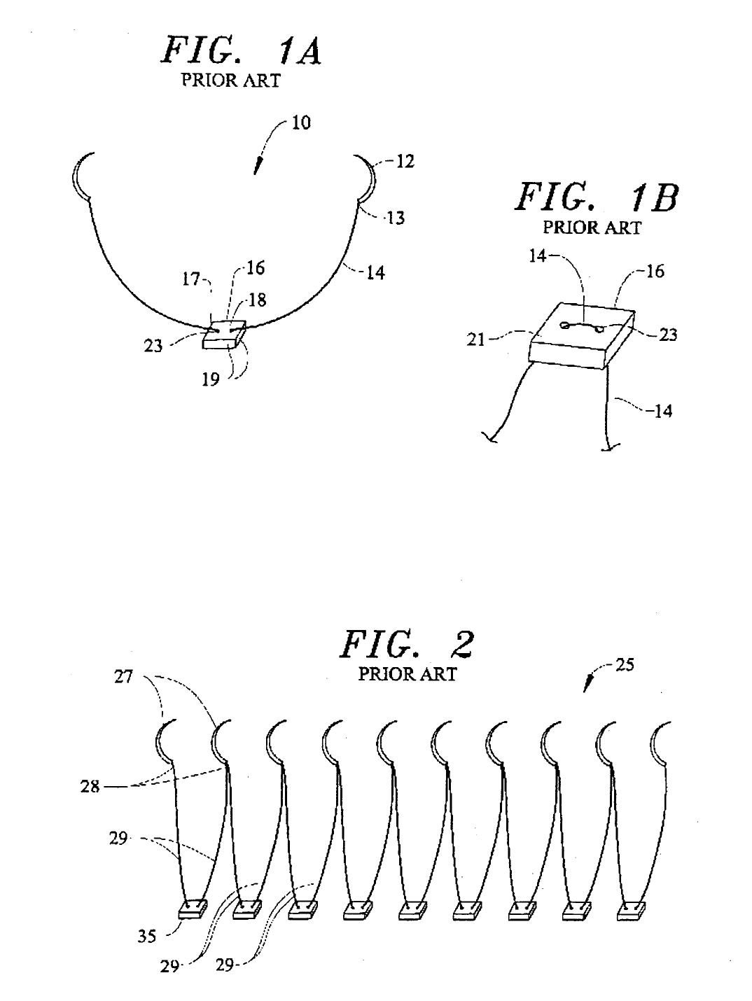

The efficacy of the needle-suture combination 100, compared to that of an example prior art needle-suture combination 10 (FIG. 1), is demonstrated through the following procedure.

Preparation of the Aortic Segment

Approximately 6 centimeter (cm) segments of the descending thoracic and upper abdominal aorta was removed from anesthetized and euthanized canines or swine. The segments were preserved either in 0.9% sodium chloride for acute usage or 10% formaldehyde for future testing. The branches of the aortic segments were either ligated with titanium clips or closed with 4-0 or 5-0 polypropylene purse-string sutures to ensure no leakage of normal saline when pressurized manually with a 20 milliliters (mL) syringe. The aortic segment was then divided in half for plegeted suture anastomosis.

Preparation of Needle-Suture Combination 100

Polytetrafluoroethylene (PTFE), or Teflon®, felt manufactured by and commercially available from Deknatel, Inc., Fall River, Mass. was used and cut into approximately 3 millimeter (mm) by 5 mm rectangular shapes for use as pledgets. Suture material used was Tev-Dek.™, manufactured by and commercially available from Genzyme Surgical Products Corp. Two 4-0 Tev-Dek™ sutures were passed in and out of the pledget using a french eye needle.

Creation and Testing of the Aortic Anastomosis

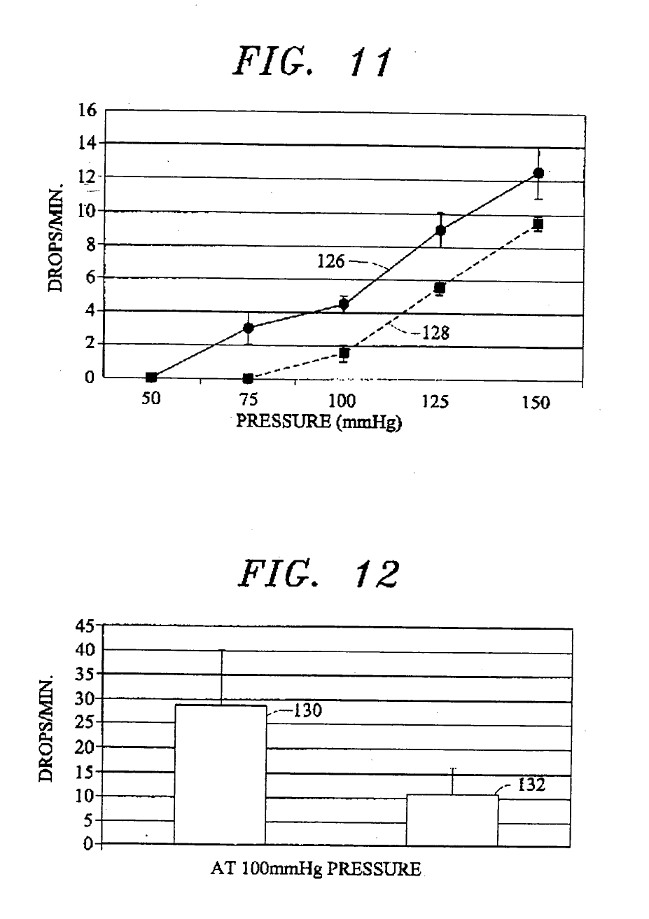

The aortic anastomosis was performed using the procedure described above with respect to FIGS. 8-10, forming a continuous horizontal “mattress” type suture configuration, as well as a creating an additional “running” suture configuration. In the test, two different studies were performed to yield the results shown in FIGS. 11 and 12. In the first study, two different aortic segments were anastomosized, first using the needle-suture combination 10 to form conventional mattress sutures, and second, using the needle-suture combination 100 to form both mattress sutures and running sutures, as described above. A leak rate was obtained over a typical range of intra-aortic pressures in a human (50 to 150 mmHg). In the second study, eight anastomoses were tested at 100 mmHg pressure for leak, using both the method and the needle-suture combination 100, and using the method and the needle-suture combination 10 for comparison.

Results

The results of the first study are summarized in the line graph of FIG. 11. The line 126 represents the leak rate over a range of pressures for aortic anastomosis performed using a method and the needle-suture combination 10 known in the prior art. The line 128 represents the leak rate over a range of pressures for aortic anastomosis performed using an embodiment of the disclosed method and the needle-suture combination 100. As can be seen from FIG. 11, the embodiment of the disclosed method, using the needle-suture combination 100, reduced the leak rate versus the conventional mattress stitch over a range of pressures (50 to 150 mmHg). The results of the second study are summarized in the bar graph of FIG. 12. In FIG. 12, the bar 130 represents the leak rate at 100 mmHg pressure for aortic anastomosis performed using a mattress stitch technique and the needle-suture combination 10 known in the prior art. The bar 132 represents the leak rate at 100 mmHg pressure for aortic anastomosis performed using the embodiment of the disclosed method and the needle-suture combination 100. As can be seen from FIG. 12, the embodiment of the disclosed method, using the needle-suture combination 100, reduced the leak rate versus the conventional mattress stitch at 100 mmHg pressure by at least approximately 17 drops/minute.

It should be emphasized that the above-described embodiments of the needle-suture combination and embodiments of method for using the needle-suture combination are merely possible example implementations. Many variations and modifications may be made to the above-described embodiment(s). All such modifications and variations are intended to be included herein within the scope of this disclosure and protected by the following claims.

Therefore, having thus described the invention, at least the following is claimed:

1. A needle-suture combination comprising:
two surgical needles, each including
an end having a pointed tip and
an end having a suture-receiving aperture; and
a first suture and a second suture attached to each of the two needles at the end having a suture-receiving aperture.

2. The needle-suture combination of claim 1, wherein the suture-receiving aperture end of the needle is swaged on both the first and second sutures.

3. The needle-suture combination of claim 1, wherein the first and second sutures are intertwined into a single fiber at each end, forming a single end portion, and
wherein the suture-receiving aperture end of the needle is swaged on the single end portion of the intertwined sutures.

4. The needle-suture combination of claim 1, wherein the first and second sutures are attached to the suture-receiving aperture end of the needle via at least one of the following: glue, cement, adhesive, ultrasonic welding, shrink-wrap tubing and combinations thereof.

5. The needle-suture combination of claim 1, wherein the first and second sutures have different colors.

6. A needle-suture combination comprising:
two surgical needles, each including an end having a suture-receiving aperture;
a first suture and a second suture attached to each of the two needles at the suture-receiving aperture end; and
a pledget member, wherein the first and second sutures are threaded through the pledget.

7. The needle-suture combination of claim 6, wherein the pledget has a shape chosen from: circular, elliptical, square, rectangular, tubular and combinations thereof.

8. The needle-suture combination of claim 6, wherein the pledget further comprises two openings therethrough.

9. The needle-suture combination of claim 8, wherein the first and second sutures are woven through the two openings.

10. The needle-suture combination of claim 6, wherein the pledget further comprises a first pair and a second pair of openings therethrough.

11. The needle-suture combination of claim 10, wherein the first suture is woven through the first pair of the openings and the second suture is threaded through the second pair of the openings.

12. The needle-suture combination of claim 6, wherein the pledget is substantially fixed along the length of the first and second sutures.

13. The needle-suture combination of claim 6, wherein the pledget is slidable along the first and second sutures.

14. The needle-suture combination of claim 6, wherein the pledget comprises at least two openings, and
wherein the first and second sutures are threaded through the openings and are tied in a knot, the knot being positioned between the openings.

15. The needle-suture combination of claim 6, wherein the pledget comprises at least two openings, and
wherein the first and second sutures are woven through the suture mounting openings and are tied in two knots, each knot being located between the openings.

16. The needle-suture combination of claim 6, wherein the pledget comprises at least two openings, and
wherein the first and second sutures are threaded through the openings and are tied in at least three knots, each of the knots being positioned between the openings.

17. A needle-suture combination comprising:
two surgical needles, each including
an end having a pointed tip and
an end having a suture-receiving aperture;
a first suture and a second suture attached to each of the two needles at the end having a suture-receiving aperture, wherein the first and second sutures have different colors, and wherein the suture-receiving aperture end of the needle is swaged on both the first and second sutures; and
a pledget member, wherein the first and second sutures are threaded through the pledget, wherein the pledget further comprises a first pair and a second pair of openings therethrough, and wherein the first suture is woven through the first pair of the openings and the second suture is threaded through the second pair of the openings and are tied in a knot, the knot being positioned between the openings.

18. A method of using a needle-suture combination, comprising:
passing through a first tissue and a second tissue two needle-suture combinations each having first and second needles connected to first and second sutures;
pulling the sutures therethrough;
disconnecting the needles from the sutures, thereby providing free ends on the sutures;
tying the free ends of the first sutures of the first needle-suture combination together; and
tying the free end of the second suture of the first needle-suture combination with the second suture of the second needle-suture combination.

19. The method of claim 18, further comprising forming a fluid-tight seal between the first tissue and the second tissue.

20. The method of claim 18, wherein the method is used to accomplish at least one of the following: heart valve replacement, aortic anastomosis, vascular grafting, ventricular wall aneurysectomy, coronary revascularization, bowel anastomosis and suturing of other circular incisions.

21. The method of claim 18, further comprising contacting the first tissue with pledgets fixed on the sutures.

22. The method of claim 21, further comprising providing counter tension with a knot in the sutures disposed between openings in the pledget.
